(19) United States
(12) Patent Application Publication
(10) Pub. No.: US 2018/0325893 A1
(43) Pub. Date: Nov. 15, 2018

(54) SUBACUTE ADMINISTRATION OF NMDA MODULATORS ALONE OR IN COMBINATION

(71) Applicant: Northwestern University, Evanston, IL (US)

(72) Inventors: Joseph R. Moskal, Evanston, IL (US); Hebert Meltzer, Evanston, IL (US)

(21) Appl. No.: 15/773,084

(22) PCT Filed: Oct. 14, 2016
(86) PCT No.: PCT/US2016/057071

§ 371 Date: May 2, 2018

Related U.S. Application Data
(60) Provisional application No. 62/242,633, filed on Oct. 16, 2015.

(51) Int. Cl. A61K 31/496; A61P 25/18; A61P 25/24

(52) U.S. Cl. CPC A61K 31/496; A61P 25/18; A61K 38/07; A61K 2300/00; A61P 25/24

(57) ABSTRACT

This disclosure features combinations of NMDA modulators and atypical antipsychotics. The disclosure provides for example, methods of treating schizophrenia, bipolar disorder, and/or cognitive impairment disorder in a patient in need thereof, comprising administering e.g., rapastinel and an atypical antipsychotic.

### CROSS REFERENCE

This application claims priority to U.S. Patent Application No. 62/242,633, filed on Oct. 16, 2015, the entire content of which is incorporated herein by reference.

### BACKGROUND

N-methyl-d-aspartate (NMDA) receptors (NMDAR) are postsynaptic, ionotropic receptors that are responsive to, inter alia, the excitatory amino acids glutamate and glycine and the synthetic compound NMDA. The NMDA receptor controls the flow of both divalent and monovalent ions into the postsynaptic neural cell through a receptor associated channel. The NMDA receptor has been implicated during development in specifying neuronal architecture and synaptic connectivity, and may be involved in experience-dependent synaptic modifications. In addition, NMDA receptors are also thought to be involved in long term potentiation and central nervous system disorders.

The NMDA receptor is believed to consist of several protein chains embedded in the postsynaptic membrane. The first two types of subunits discovered so far form a large extracellular region, which probably contains most of the allosteric binding sites, several transmembrane regions looped and folded so as to form a pore or channel, which is permeable to Ca⁺⁺, and a carboxyl terminal region. The opening and closing of the channel is regulated by the binding of various ligands to domains (allosteric sites) of the protein residing on the extracellular surface. The binding of the ligands is thought to affect a conformational change in the overall structure of the protein which is ultimately reflected in the channel opening, partially opening, partially closing, or closing.

Recently, improved partial agonist of NMDAR such as rapastinel has been reported to enhance hippocampal-dependent spatial learning tasks in rodents, indicating it may have cognitive enhancing, as well as antidepressant properties. Rapastinel is exemplified by the following structure:

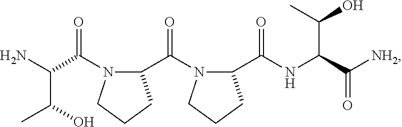

with a molecular weight: 413.47, and a chemical formula: C₁₈H₃₁N₅O₆. Rapastinel exhibits nootropic, neuroprotective and antinociceptive activity, and enhances learning, memory and cognition in vivo.

Ketamine, an NMDAR non-competitive antagonist, has also been reported to produce rapidly acting antidepressant properties; however, it also causes dissociative and psychotic-like effects, as well as cognitive impairment, in healthy humans, and exacerbates psychosis, but not cognitive impairment, in patients with schizophrenia. Ketamine also causes deficits in cognition in rodents, including novel object recognition (NOR), an analog of human declarative memory. NOR is dependent on the integrated action of the hippocampus, entorhinal, perirhinal and temporal association cortices, and prefrontal cortex. Glutamate, via its actions at NMDAR and α-amino-3-hydroxy-5-methyl-4-isoxazolepropionic acid (AMPA) receptors, has a profound effect on synaptic plasticity and, thus, plays a major role in learning and memory.

There continues to be a serious need for therapies, e.g., medications that can be administered alone or in conjunction with other active agents to treat depression, such as patients with bipolar depression, and/or to treat patients having schizophrenia.

### SUMMARY

This disclosure features in part combinations that include one or more atypical antipsychotics and an NMDA modulator, such rapastinel and other NMDA modulators disclosed herein (each of which is sometimes referred to herein as a “component”). The beneficial effects of such a combination are based, in part, on the finding that administration of rapastinel and an atypical antipsychotic (e.g. lurasidone) (e.g., a sub acute dose) can reverse and/or prevent NMDAR antagonist-induced cognitive impairment (e.g., NMDAR antagonist-induced impairment in novel object recognition; e.g., induced through repeated dosing of the NMDAR antagonist). Disclosed combinations can further include one or more other biologically active ingredients (e.g., one or more other anti-depressant compounds) and/or one or more pharmaceutically acceptable excipients and/or carriers. The components of the combination (sometimes also referred to herein as chemical entities or chemical compounds) can be administered to a patient in a sequential manner (each component is administered at a different time) or in a substantially simultaneous manner. It will be appreciated that the components may be present in the same pharmaceutically acceptable carrier and, therefore, administered simultaneously. Alternatively, each of the components can be present in separate pharmaceutical carriers, such as, conventional oral dosage forms, or parenteral forms, (or one component may be oral and the other parenteral) that can be administered either simultaneously or sequentially. Accordingly, in one aspect, methods of substantially reversing or preventing cognitive impairment in a patient acutely administered a NMDAR antagonist are provided, which include administering a disclosed NMDA modulator such as rapastine and an atypical antipsychotic (e.g. lurasidone). For example, provided herein is a method of treating schizophrenia or bipolar disorder in a patient in need thereof, comprising administering to the patient: an atypical antipsychotic; and an NMDA modulator selected from the group consisting of rapastinel, (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide. In certain contemplated methods, the NMDA modulator and the atypical antipsychotic are each administered at a dose that is sub-effective if administered alone.

In another embodiment, provided here is a pharmaceutically acceptable composition comprising an NMDA modulator e.g., rapastinel and an atypical antipsychotic (e.g., lurasidone). For example, such a composition may include sub-acute or sub-effective doses (based on administration of the drug alone) of rapastinel and/or lurasidone.

### BRIEF DESCRIPTION OF THE DRAWINGS

FIG. 1 shows acute ketamine (ket; 30 mg/kg) but not rapastinel (rap; 1.0 mg/kg) induced a significant NOR deficit in male C57BL/6J mice. Rap (1.0 mg/kg) significantly prevented the ket-induced NOR deficit ($$$P<0.001).

FIG. 2 shows rap (1.0 mg/kg) significantly reversed subchronic (sc) PCP & let (10 and 30 mg/kg; i.p.; b.i.d.; 7 days; followed by 7 days washout)-induced NOR deficit (###P<0.001).

FIG. 3 shows sub-effective dose (SED) acute rapastinel plus SED lurasidone (lur) significantly reversed scket-induced NOR deficit (##P<0.001) but neither drug above at these doses was effective.

### DETAILED DESCRIPTION

This disclosure features combinations that include one or more comprising administering an NMDA modulator such as described herein, e.g., rapastinel, and an atypical antipsychotic (each of which is sometimes referred to herein as a “component”). The beneficial effects of the combination are based, in part, on the finding that administration of a NMDA modulator such as rapastinel (e.g., a sub-acute or sub-effective dose) together with an atypical antipsychotic (e.g. lurasidone) can reverse and/or prevent cognitive impairment (e.g., NMDAR antagonist-induced cognitive impairment) in novel object recognition; e.g., induced through repeated dosing of the NMDAR antagonist). The combinations can further include one or more other biologically active ingredients (e.g., one or more other anti-depressant compounds) and/or one or more pharmaceutically acceptable excipients and/or carriers. The components of the combination (sometimes also referred to herein as chemical entities or chemical compounds) can be administered to a patient in a sequential manner (each component is administered at a different time) or in a substantially simultaneous manner. It will be appreciated that the components may be present in the same pharmaceutically acceptable carrier and, therefore, administered simultaneously. Alternatively, each of the components can be present in separate pharmaceutical carriers, such as, conventional oral dosage forms, or parenteral forms, (or one component may be oral and the other parenteral) that can be administered either simultaneously or sequentially. In some embodiments, pre-treatment or substantially co-administration of an atypical antipsychotic) with rapastinel (or e.g., a disclosed NMDA modulator) (i.e., given prior to the administration of one or more atypical antipsychotics) can be particularly beneficial.

### Definitions

“Rapastinel” (or “GLYX-13”) is represented by the following formula:

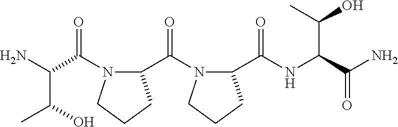

and includes polymorphs, hydrates, solvates, free bases, and/or suitable salt forms of the above compound.

“Treating” includes any effect, e.g., lessening, reducing, modulating, or eliminating, that results in the improvement of the condition, disease, disorder and the like.

As used herein, the terms “NMDA receptor antagonist” and “NMDAR antagonist” generally both refer to a chemical entity that is capable of binding to a glycine binding site of an NMDA receptor rand works to antagonize, or inhibit, the action of the N-Methyl-D-aspartate receptor (NMDAR).

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions.

The term “pharmaceutical composition” as used herein refers to a composition comprising at least one of the components of the combinations disclosed herein formulated together with one or more pharmaceutically acceptable carriers and/or excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The combinations of the invention can be administered as described herein to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). In some embodiments, the mammal treated in the methods of the invention is a mammal in which treatment e.g., of depression is desired.

The term “pharmaceutically acceptable salt(s)” as used herein refers to salts of acidic or basic groups that may be present in compounds used in the present combinations. Compounds included in the present combinations that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present combinations that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Compounds included in the present combinations that include a basic or acidic moiety may also form pharmaceutically acceptable salts with various amino acids. Compounds included in the present combinations may contain both acidic and basic groups; for example, one amino and one carboxylic acid group. In such a case, the compound can exist as an acid addition salt, a zwitterion, or a base salt. Throughout this disclosure a disclosed compound also may encompass a pharmaceutically acceptable salt.

### Combination Components

Rapastinel may be obtained by well-known recombinant or synthetic methods such as those described in U.S. Pat. Nos. 5,763,393 and 4,086,196 herein incorporated by reference. Also contemplated are polymorphs, hydrates, homologs, solvates, free bases, and/or suitable salt forms of rapastinel such as, but not limited to, the acetate salt. The peptide may be in cyclized or non-cyclized form as further described in U.S. Pat. No. 5,763,393. In some embodiments, a rapastinel analog may include an insertion or deletion of a moiety on one or more of the Thr or Pro groups such as a deletion of CH₃, OH, or NH₂ moiety. In other embodiments, rapastinel may be optionally substituted with one or more halogens, C₁-C₃ alkyl (optionally substituted with halogen or amino), hydroxyl, and/or amino. Other compounds contemplated for use herein include Glycine-site partial agonists of the NMDAR disclosed in U.S. Pat. No. 5,763,393, U.S. Pat. No. 6,107,271, and Wood et al., Neuro. Report, 19, 1059-1061, 2008, the entire contents of which are herein incorporated by reference.

It may be understood that the peptides disclosed here can include both natural and unnatural amino acids, e.g., all natural amino acids (or derivatives thereof), all unnatural amino acids (or derivatives thereof), or a mixture of natural and unnatural amino acids. For example, one, two, three or more of the amino acids in rapastinel may each have, independently, a D- or L-configuration.

Contemplated NMDA modulators also include (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

Contemplated atypical antipsychotics include lurasidone, quetiapine, olanzapine, asenapine, risperidone, ziprasidone, clozapine, melperone, cariprazien, aripiprazole, pimavenserin, ITI-007, RP506, and reomxipride.

In some embodiments, a NMDAR antagonist is selected from the group consisting of ketamine, memantine, lanicemine (AZD6765), CERC-301, dextromethorphan, dextrorphan, phencyclidine, dizocilpine (MK-801), amantadine, ifenprodil, AV-101, AZD 6423, and riluzole, or a pharmaceutically acceptable salt or prodrug thereof. Also contemplated are derivatives of the aforementioned NMDAR antagonists. NMDAR antagonists may be selected, in an embodiment, from the group consisting of nitrous oxide, atomoxetine, dextrallorphan, diphenidine, eticyclidine, gacyclidine, ibogaine, methoxetamine, nitromemantine, rolicyclidine, tenocyclidine, methoxydine, tiletamine, neramexane, eliprodil, etoxadrol, dexoxadrol, methadone, WMS-2539, NEFA, remacemide, delucemine, 8A-PDHQ, aptiganel (Cerestat, CNS-1102), HU-211, remacemide, rhynchophylline, TK-40, Traxoprodil (CP-101,606), 1-Aminocyclopropanecarboxylic acid (ACPC), kynurenic acid or a derivative thereof, 2-carboxytetrahydroquinoline or a derivative thereof, 2-carboxyindole or a derivative thereof, 4-hydroxy-2-quinoline or a derivative thereof, 4-hydroxyquinoline or a derivative thereof, quinoxaline-2,3-dione or a derivative thereof, trycyclic antagonists, lacosamide, L-phenylalanine, midafotel, and aptiganel, or a pharmaceutically acceptable salt or prodrug thereof.

### Methods

In one aspect, methods of substantially reversing or preventing cognitive impairment disorder in a patient comprising administering an atypical antipsychotic (e.g. such as selected from the group consisting of lurasidone, quetiapine, olanzapine, asenapine, risperidone, ziprasidone, clozapine, melperone, cariprazien, aripiprazole, pimavenserin, ITI-007, RP506, and reomxipride) and an NMDA modulator such as described herein, e.g., rapastinel, are provided.

In another aspect, methods of treating a cognitive impairment disorder in a patient in need thereof are provided, which include administering an amount of an NMDA modulator such as described herein, e.g., rapastinel, and an atypical antipsychotic. The cognitive impairment disorder can be due to one or more of: deficit in cognitive ability, congenital defect, environmental factor(s), or drug induced and include, but are not limited to, learning disorders and/or dyslexia. In some embodiments, an administration of amount of rapastinel (e.g., a subacute amount) occurs before or after one or more atypical antipsychotics are administered. In other embodiments, administration of an NMDA modulator such as described herein, e.g., rapastinel, occurs substantially simultaneously with administration of one or more of atypical antipsychotics.

In a further aspect, methods of treating a disorder, condition, or disease including, but not limited to: neurological or other disorders (e.g., stroke, psychotic disorder, pain (e.g., neuropathic pain), depression (e.g., major depression), Parkinson's disease, and Alzheimer's disease); a central nervous system disease (e.g., neurodegenerative disease, stroke, traumatic brain injury, and spinal cord injury); schizophrenia; and/or depression (e.g., refractory depression), are provided, which include administering the combinations described herein, e.g., an amount of an NMDA modulator such as described herein, e.g., rapastinel, and one or more atypical antipsychotics. Other exemplary conditions include, but are not limited to, learning disorder, autistic disorder, attention-deficit hyperactivity disorder, anxiety, migraine, Tourette's syndrome, phobia, post-traumatic stress disorder, dementia, memory deficits associated with aging, AIDS dementia, Huntington's disease, spasticity, myoclonus, muscle spasm, bipolar disorder, neuropathic pain, substance abuse disorder, urinary incontinence, ischemia, special learning disorders, seizures, post-stroke convulsions, brain ischemia, hypoglycemia, cardiac arrest and epilepsy. In some embodiments, rapastinel and one or more atypical antipsychotics are administered substantially simultaneously. In other embodiments, rapastinel and one or more atypical antipsychotics are administered sequentially, e.g., rapastinel is administered before or after one or more atypical antipsychotics.

Contemplated methods include a method of treating autism and/or an autism spectrum disorder in a patient in need thereof, which include administering the combinations described herein, e.g., an amount of an NMDA modulator such as described herein, e.g., rapastinel, and one or more of atypical antipsychotics. In an embodiment, a method for reducing the symptoms of autism in a patient in need thereof is contemplated, comprising administering the combinations described herein, e.g., an amount of an NMDA modulator such as described herein, e.g., rapastinel, and one or more atypical antipsychotics. For example, upon administration, the combinations may decrease the incidence of one or more symptoms of autism such as eye contact avoidance, failure to socialize, attention deficit, poor mood, hyperactivity, abnormal sound sensitivity, inappropriate speech, disrupted sleep, and perseveration. Such decreased incidence may be measured relative to the incidence in the untreated individual or an untreated individual(s).

In some embodiments, patients suffering from autism also suffer from another medical condition, such as Fragile X syndrome, tuberous sclerosis, congenital rubella syndrome, and untreated phenylketonuria.

In some embodiments, methods of treating a disorder in a patient in need thereof are contemplated, wherein the disorder is selected from group consisting of: cerebral ischemia, stroke, brain trauma, brain tumors, acute neuropathic pain, chronic neuropathic pain, sleep disorders, drug addiction, depression, certain vision disorders, ethanol withdrawal, anxiety, memory and learning disabilities, autism, epilepsy, AIDS dementia, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, peripheral neuropathy, myelopathy, ischemic retinopathy, diabetic retinopathy, glaucoma, cardiac arrest, behavior disorders, impulse control disorders, Alzheimer's disease, memory loss that accompanies early stage Alzheimer's disease, attention deficit disorder, ADHD, schizophrenia, amelioration of opiate, nicotine addiction, ethanol addition, traumatic brain injury, spinal cord injury, post-traumatic stress syndrome, and Huntington's chorea that includes administering the combinations described herein, e.g., an amount of an NMDA modulator such as described herein, e.g., rapastinel, and one or more NMDAR antagonists.

In some embodiments, contemplated herein are methods of treating attention deficit disorder, ADHD (attention deficit hyperactivity disorder), schizophrenia, anxiety, amelioration of opiate, nicotine and/or ethanol addiction (e.g., method of treating such addiction or ameliorating the side effects of withdrawing from such addiction), spinal cord injury diabetic retinopathy, traumatic brain injury, post-traumatic stress syndrome and/or Huntington's chorea, in a patient in need thereof, that includes administering the combinations described herein, e.g., an amount of an NMDA modulator such as described herein, e.g., rapastinel, and one or more of atypical antipsychotics. For example, patients suffering from schizophrenia, addiction (e.g. ethanol or opiate), autism, Huntington's chorea, traumatic brain injury, spinal cord injury, post-traumatic stress syndrome and diabetic retinopathy may all be suffering from altered NMDA receptor expression or functions.

For example, provided herein is a method of treating depression in a patient need thereof, comprising administering the combinations described herein, e.g., an amount of an NMDA modulator such as described herein, e.g., rapastinel, and one or more of an atypical antipsychotic. In certain embodiments, the treatment-resistant patient is identified as one who has been treated with at least two types of antidepressant treatments prior to administration of the combinations described herein. In other embodiments, the treatment-resistant patient is one who is identified as unwilling or unable to tolerate a side effect of at least one type of antidepressant treatment.

The most common depression conditions include major depressive disorder and dysthymic disorder. Other depression conditions develop under unique circumstances. Such depression conditions include but are not limited to psychotic depression, postpartum depression, seasonal affective disorder (SAD), mood disorder, depressions caused by chronic medical conditions such as cancer or chronic pain, chemotherapy, chronic stress, post traumatic stress disorders, and bipolar disorder (or manic depressive disorder).

Refractory depression occurs in patients suffering from depression who are resistant to standard pharmacological treatments, including tricyclic antidepressants, MAOIs, SSRIs, and double and triple uptake inhibitors and/or anxiolytic drugs, as well non-pharmacological treatments such as psychotherapy, electroconvulsive therapy, vagus nerve stimulation and/or transcranial magnetic stimulation. A treatment resistant-patient may be identified as one who fails to experience alleviation of one or more symptoms of depression (e.g., persistent anxious or sad feelings, feelings of helplessness, hopelessness, pessimism) despite undergoing one or more standard pharmacological or non-pharmacological treatments. In certain embodiments, a treatment-resistant patient is one who fails to experience alleviation of one or more symptoms of depression despite undergoing treatment with two different antidepressant drugs. In other embodiments, a treatment-resistant patient is one who fails to experience alleviation of one or more symptoms of depression despite undergoing treatment with four different antidepressant drugs. A treatment-resistant patient may also be identified as one who is unwilling or unable to tolerate the side effects of one or more standard pharmacological or non-pharmacological treatments.

For example, contemplated herein are methods for treating a subset of depressed patients, e.g. those with unipolar or bipolar depression with psychotic features who e.g., may be prone to experience psychosis. Thus, contemplated patients with mood disorders may be at higher risk for additional cognitive impairment or recurrence of psychosis from ketamine than from rapastinel, even with a single treatment, as the adverse effects of ketamine on cognition and psychopathology may emerge from a single administration at doses comparable to those which are required to treat depression. With multiple administrations of ketamine for persistent, recurrent treatment of resistant depression, as has sometimes been done with ketamine, the risk for cognitive impairment and psychosis could be higher. Contemplated herein for example, is administration of sub-effective dose rapastinel which may potentiate sub-effective dose of multi-receptor AAPD/antidepressant drug lurasidone. In an embodiment, atypical AAPDs such as lurasidone, may reduce the risk for cognitive impairment and psychosis in treatment-resistant depressed patients (e.g., being treated with ketamine).

In yet another aspect, a method for enhancing pain relief and for providing analgesia to an animal is provided. In some embodiments, methods are provided for treating neuropathic pain. The neuropathic pain may be acute or chronic. In some cases, the neuropathic pain may be associated with a condition such as herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, and cancer chemotherapeutic-induced neuropathic pain. Methods for enhancing pain relief and for providing analgesia to a patient are also contemplated.

In certain embodiments, methods for treating schizophrenia are provided. For example, paranoid type schizophrenia, disorganized type schizophrenia (i.e., hebephrenic schizophrenia), catatonic type schizophrenia, undifferentiated type schizophrenia, residual type schizophrenia, post-schizophrenic depression, and simple schizophrenia may be treated using the methods and compositions contemplated herein. Psychotic disorders such as schizoaffective disorders, delusional disorders, brief psychotic disorders, shared psychotic disorders, and psychotic disorders with delusions or hallucinations may also be treated using the compositions contemplated herein.

Paranoid schizophrenia may be characterized where delusions or auditory hallucinations are present, but thought disorder, disorganized behavior, or affective flattening are not. Delusions may be persecutory and/or grandiose, but in addition to these, other themes such as jealousy, religiosity, or somatization may also be present.

Disorganized type schizophrenia may be characterized where thought disorder and flat affect are present together.

Catatonic type schizophrenia may be characterized where the subject may be almost immobile or exhibit agitated, purposeless movement. Symptoms can include catatonic stupor and waxy flexibility.

Undifferentiated type schizophrenia may be characterized where psychotic symptoms are present but the criteria for paranoid, disorganized, or catatonic types have not been met.

Residual type schizophrenia may be characterized where positive symptoms are present at a low intensity only.

Post-schizophrenic depression may be characterized where a depressive episode arises in the aftermath of a schizophrenic illness where some low-level schizophrenic symptoms may still be present.

Simple schizophrenia may be characterized by insidious and progressive development of prominent negative symptoms with no history of psychotic episodes.

In some embodiments, methods are provided for treating psychotic symptoms that may be present in other mental disorders, including, but not limited to, bipolar disorder, borderline personality disorder, drug intoxication, and drug-induced psychosis.

In another embodiment, methods for treating delusions (e.g., “non-bizarre”) that may be present in, for example, delusional disorder are provided.

Also provided are methods for treating social withdrawal in conditions including, but not limited to, social anxiety disorder, avoidant personality disorder, and schizotypal personality disorder.

Additionally, methods are provided for treating obsessive-compulsive disorder (OCD).

In another embodiment, a method of treating Alzheimer's disease, or e.g., treatment of memory loss that e.g., accompanies early stage Alzheimer's disease, in a patient in need thereof is provided, comprising administering the combinations described herein, e.g., an amount of an NMDA modulator such as described herein, e.g., rapastinel, and one or more atypical antipsychotics. Also provided herein is a method of modulating an Alzheimer's amyloid protein (e.g., beta amyloid peptide, e.g. the isoform AJ3₁-4₂), in-vitro or in-vivo (e.g. in a cell) comprising contacting the protein with the combinations described herein, e.g., an amount of an NMDA modulator such as described herein, e.g., rapastinel, and one or more atypical antipsychotics. For example, in some embodiments, rapastinel or another disclosed compound may block the ability of such amyloid protein to inhibit long-term potentiation in hippocampal slices as well as apoptotic neuronal cell death. In some embodiments, a disclosed compound (e.g., rapastinel) may provide neuroprotective properties to a Alzheimer's patient in need thereof, for example, may provide a therapeutic effect on later stage Alzheimer's-associated neuronal cell death.

In some embodiments, the patient is a human, e.g. a human pediatric patient.

The present disclosure contemplates “combination therapy,” which includes (but is not limited to) co-administering an amount of an NMDA modulator such as described herein, e.g., rapastinel, and one or more atypical antipsychotics, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, one or more of the components of the combinations described herein may be administered parenterally to a patient including, but not limited to, subcutaneously and intravenously. In some embodiments, one or more of the components of the combinations described herein may also be administered via slow controlled i.v. infusion or by release from an implant device. In some embodiments, a patient has substantial improvement in, e.g., cognitive impairment, after 1 hour, 2 hours 4 hours, 8 hours, 12 hours, 1 day, 2 days, 3 days, 4 days, 5 days, 6 days, 1 week, or even after 8 days of a one (single) dose administration of rapastinel.

A therapeutically effective amount of a disclosed compound required for use in therapy varies with the nature of the condition being treated, the length of treatment time desired, the age and the condition of the patient, and is ultimately determined by the attending physician. In general, however, doses employed for adult human treatment typically are in the range of about 0.01 mg/kg to about 1000 mg/kg per day (e.g., about 0.01 mg/kg to about 100 mg/kg per day, about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day) of each component of the combinations described herein. In certain embodiments, doses of rapastinel employed for adult human treatment typically are in the range of about 0.01 mg/kg to about 100 mg/kg per day (e.g., about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day, about 1 mg/kg per day). The desired dose may be conveniently administered in a single dose, or as multiple doses administered at appropriate intervals, for example as two, three, four or more sub-doses per day.

A number of factors may lead to each component of the combinations described herein being administered over a wide range of dosages. When given in combination with other therapeutic agents, the dosage of the compounds of the present invention may be given at relatively lower dosages. In certain embodiments, the dosage of rapastinel may be from about 1 ng/kg to about 100 mg/kg. The dosage of rapastinel may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the disclosed compound, e.g. rapastinel, may be dosed at amount that reverses or prevents cognitive impairment.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water (e.g., water for injection).

In some embodiments, disclosed compounds, e.g. rapastinel, may be provided as part of an aqueous composition that is suitable for intravenous injection. In certain embodiments, such compositions can include: (i) 60 mg/mL to about 200 mg/mL (e.g., about 125 mg/mL to about 175 mg/mL; e.g., about 150 mg/mL or about 75 mg/mL) of a pharmaceutically active compound having the formula:

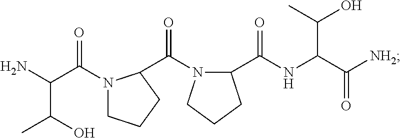 [SMILES: CC(O)C(N)C(=O)N1CCCC1C(=O)N1CCCC1C(=O)NC(C(N)=O)C(C)O]

OH; or a pharmaceutically acceptable salt thereof; (ii) water (e.g., water for injection); and (iii) an acid; wherein the stable, aqueous composition has a pH of from about 3.9 to about 5.5 (e.g., from about 4.0 to about 5.0, from about 4.2 to about 5.0, from about 4.1 to about 4.7, from about 4.2 to about 4.8, about 4.0, about 4.5) at 25° C. In certain embodiments, such compositions can be disposed within a receptacle (e.g., a prefilled syringe or vial), in which the amount of the compound is extractable as at least one single dose. In certain embodiments, the single dose can have a volume of about 1 mL to about 4 mL (e.g., 3 mL).

In certain embodiments, the aqueous compositions can include about 200 mg to about 500 mg (e.g., about 450 mg; about 375; or about 225 mg) of the pharmaceutically active compound.

In certain embodiments, the acid can be selected from the group consisting of fumaric acid, malic acid, lactic acid, hydrochloric acid, hydrobromic acid, acetic acid, citric acid, phosphoric acid, nitric acid, sulfuric acid, and ascorbic acid. In certain embodiments, the acid provides chloride ions in the aqueous composition (e.g., hydrochloric acid).

### EXAMPLES

### Materials and Methods for Examples 1-3

Three cohorts of male C57BL/6J mice (2½-3 month old, Jackson, Mass., USA), N=40, 45, and 48 were used in Examples 1, 2, 3 respectively. Mice were group housed (five/cage) in a controlled environment held at 21±2° C. and 50±15% relative humidity with a 14:10 h light-dark period (lights on: 05:00 am). All experiments were conducted during the light phase. Food and water were available ad libitum. The mice were habituated to the colony upon arrival for a week, during which time, they were not handled. All experiments were conducted in accordance with Institutional Animal Care and Use Committee of Northwestern University, Chicago.

Rapastinel was obtained from SAI Life Sciences (India). PCP was a generous gift from National Institute of Drug Abuse. Ketamine was purchased from Sigma Aldrich (St. Louis, Mo.). Lurasidone was provided by Sumitomo Dainippon Pharma Co., Ltd. (Osaka, Japan). Rapastinel, PCP, and ketamine were dissolved in 0.9% sterile saline (Sal). PCP and ketamine were administered intraperitoneally (ip), at a volume of 10 mL/kg body weight. Rapastinel was given intravenously (iv). The dose of rapastinel (1.0 mg/kg) was chosen because it produced optimal enhancement in learning in both young adult and aged rats and in a trace eye blink conditioning task in rabbits. The doses for ketamine (30 mg/kg) and PCP (10 mg/kg) were chosen based on prior studies, which showed that these doses induce significant cognitive impairment in mice and rats. The dose for lurasidone (0.1 mg/kg) was chosen based on prior NOR studies in C57BL/6J mice which determined the effective dose of lurasidone to restore NOR in subchronic PCP-treated mice.

For acute drug treatments, rapastinel (1.0 mg/kg, iv), lurasidone (0.1 mg/kg, i.p.) or ketamine (30 mg/kg, ip) were administered 30 min prior to the acquisition trial of the NOR task (described below) to the subchronic ketamine or subchronic PCP-treated animals. For subchronic drug treatments, 7-10 mice/cohort were randomly assigned to Sal, PCP, or ketamine. The Sal-treated mice received 0.9% NaCl; the drug treatment groups received either PCP (10 mg/kg; ip), or ketamine (30 mg/kg; ip) twice daily for 7 days. This was followed by a 7 day washout period during which time, mice were left undisturbed in the home cage until initiation of habituation (see below).

NOR testing in mice was slightly modified from Hashimoto et al (Hashimoto K.; Fujita Y.; Shimizu E.; Iyo M. European Journal of Pharmacology 2005, 519:114-7). (i.e. size of the box, usage of white background to the walls of the box instead of black reflective surfaces, and duration of the trials) based on prelimary experiments showing that when black reflective surfaces were used for the inner surfaces of the NOR box, the animals failed to explore much. Similar observations were made when large objects were used. Hence, white walls for the box and small objects for exploration were used. The dimensions of the NOR box used for mice is comparable to that of rats. It was observed that C57BL/6J mice explored less when the duration of trials were 3 or 5. Hence, animals were allowed to explore for 10 min in both trials and noticed significant increase in exploration times, and a longer duration of the trials was used. The NOR apparatus consisted of an open box made of Plexiglas (52 cm L; 52 cm W; 31 cm H) with white walls and a solid floor. The box was positioned approximately 30 cm above the floor, centered on a table such that the overhead lights could not provide a spatial cue. One day after the 7 day washout from subchronic drug treatment or Sal, mice were habituated to the empty NOR arena, as a group, for one hour, on each of three days prior to the acquisition trial. During the acquisition trial, the mice were allowed to explore two identical objects (e.g. A1 and A2) for 10 min. This was followed by a 24-hour inter-trial interval, after which the mice were returned to the home cage. During the retention trial, the mice were allowed to explore the familiar object (A) from the acquisition trial and a novel object (e.g. B). The location of the novel object in the retention trial was randomly assigned for each mouse tested using a pseudorandom schedule. The pseudorandom sequences followed the criteria suggested by Gellerman (1933) to reduce the effects of object and place preference. Also, to avoid bias or olfactory trails, objects were used in triplicates, i.e. that the same object that was used in the acquisition trial was not presented in retention trial. Behavior was recorded on video for blind scoring of object exploration. Object exploration was defined as an animal licking, sniffing, or touching the object with the forepaws while sniffing. The exploration time (s) of each object in each trial was recorded manually by the use of two stopwatches, and if the mice failed to explore for >1 (s) in both acquisition and retention trials, they were excluded from the analysis. The discrimination index (DI) [(time spent exploring the novel object—time spent exploring the familiar object)/total exploration time] was then calculated for retention trials.

All data are expressed as the mean±S.E.M. (n=7-10 per group). Exploration data were analyzed by a repeated measures analysis of variance (ANOVA) followed by the pair-wise comparison when a significant effect was detected by the ANOVA. DI data were analyzed by one-way ANOVA followed by Bonferroni test when a significant effect was detected by ANOVA.

### Example 1 Rapastinel Prevented Acute Ketamine-Induced NOR Deficit in Male C57BL/6J Mice

No significant effect on object exploration was found during acquisition trials in any of the groups (F_{3, 31}=0.90; P=0.96; data not shown). In the retention trials, there was a significant interaction between drug treatment and object exploration time (F_{3, 31}=24.76; ***P<0.001; data not shown). Further post-hoc analysis revealed that wild-type mice given sal and sal plus rapastinel (1.0 mg/kg) showed a clear preference for the novel compared to the familiar object, i.e. spent significantly more time exploring the novel versus familiar object (P<0.001). This effect was abolished in mice treated with acute ketamine (30 mg/kg)—i.e. these mice spent similar amount of time exploring both objects. Furthermore, mice given rapastinel (1.0 mg/kg) prior to acute ketamine (30 mg/kg) showed clear preference for the novel compared to the familiar object (P<0.01).

No significant effect was observed between groups in the total exploration times (acquisition trial+retention trial). Mice from all treatment groups spent almost equal times exploring in the acquisition and retention trials (sal+sal=72.5±5.7; rap+sal=76.8 76.8±3.8; ket+sal=82.4±11.0; rap+ket=79.2±8.3).

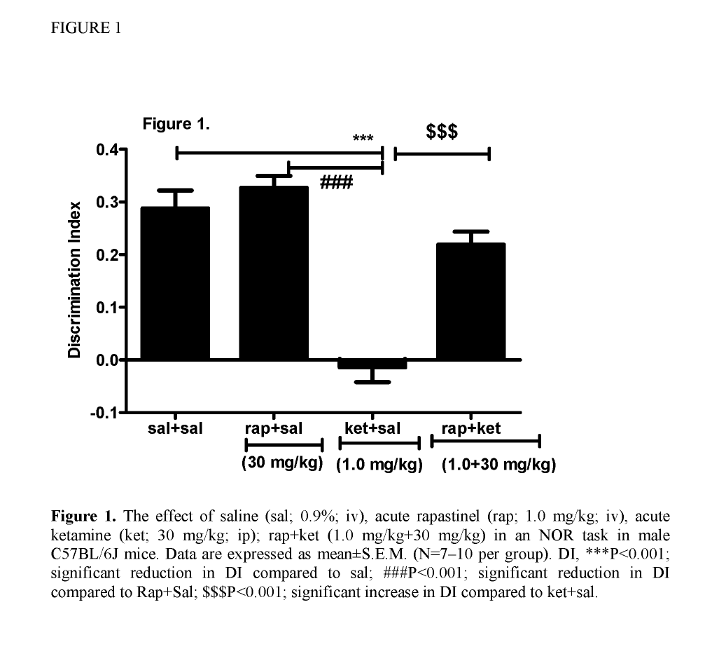

In the DI, there was a significant interaction between the groups (F_{3, 31}=28.23;***P<0.001; FIG. 1). The DI for acute ketamine plus sal-treated mice was significantly reduced compared to the sal plus sal, sal plus rapastinel, and rapastinel (1.0 mg/kg) plus ketamine (30 mg/kg) treated mice (***P<0.001; ###P<0.001; $$$P<0.001; FIG. 1). Thus, rapastinel, 1.0 mg/kg significantly prevented the acute ketamine-induced decrease in DI.

### Example 2 Rapastinel Significantly Reversed Subchronic PCP- and Subchronic Ketamine-Induced NOR Deficit in C57BL/6J Mice

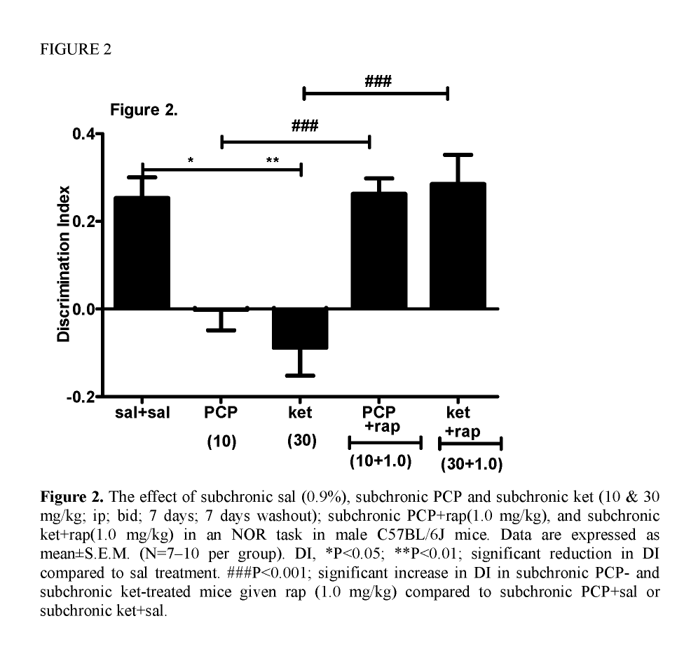

No significant effect on object exploration was found during the acquisition trial for any of the groups (F_{4, 47}=0.76; P=0.23; data not shown). In the retention trial, there was a significant interaction between drug treatment and object exploration time (F_{4, 47}=10.45; ***P<0.001; data not shown). Further post-hoc analysis revealed that wild-type mice given sal showed a clear preference for the novel compared to the familiar object (P<0.001). This effect was abolished in mice treated with subchronic ketamine or subchronic PCP treated animals. Acute rapastinel (1.0 mg/kg) treated animals explored the novel object significantly more compared to the familiar object, thereby reversing the deficit induced by subchronic PCP or subchronic ketamine (P<0.001; data not shown). In the DI, there was a significant interaction between the groups (F_{4, 47}=9.30;***P<0.001; FIG. 2). The DI for subchronic PCP— and subchronic ketamine-treated mice was significantly reduced compared to the sal plus sal treated control mice (*P<0.05 and **P<0.01; FIG. 2). The DI for subchronic PCP and subchronic ketamine treated animals given rapastinel, 1.0 mg/kg, was significantly increased, thereby significantly showing reversal of NOR impairment produced by both subchronic PCP and subchronic ketamine-treatment (###P<0.001; FIG. 2).

Total Exploration Times:

No significant effect was observed between groups in the total exploration times (acquisition trial+retention trial). Mice from all treatment groups spent almost equal times exploring in the acquisition and retention trials (sal+sal=74.5±5.7; subchronic PCP=74.8±5.5; subchronic ketamine=80.3±7.2; subchronic PCP+rapastinel=73.8±3.8; subchronic ketamine+rapastinel=86.2±8.3).

These results suggest that rapastinel differs from the effects of NMDAR non-competitive antagonists such as PCP, ketamine, and dizocilpine (MK-801) in not impairing NOR in mice.

### Example 3 Sub-Effective Dose Rapastinel Plus Sub-Effective Dose Lurasidone Reversed the Subchronic Ketamine-Induced NOR Deficit

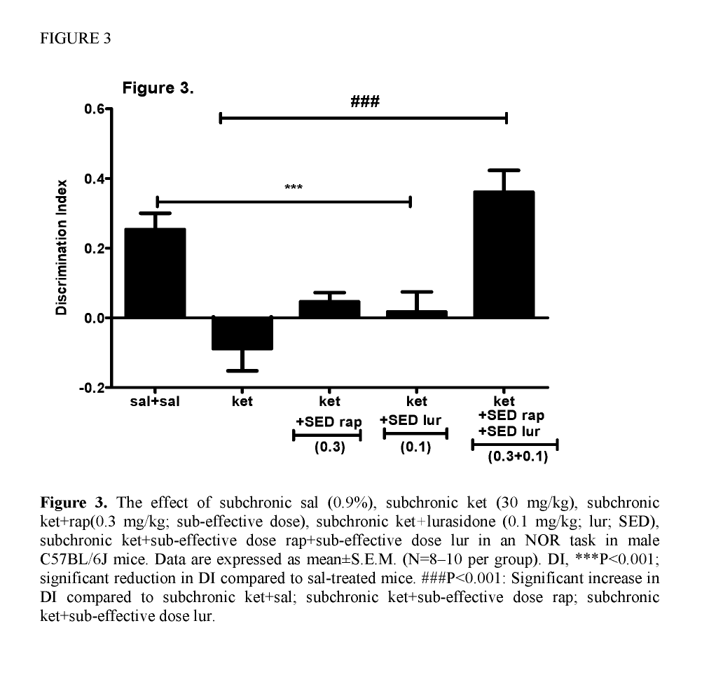

No significant effect on object exploration was found during the acquisition trial for any of the groups (F_{4, 43}=0.92; P=0.13; data not shown). In the retention trial, there was a significant interaction between drug treatment and object exploration time (F_{4, 43}=12.45; ***P<0.001; data not shown). Further post-hoc analysis revealed that wild-type mice given sal showed a clear preference for the novel compared to the familiar object (P<0.001). This effect was abolished in mice treated with scketamine and in animals given scketamine plus sub-effective dose rapastinel (0.3 mg/kg) and scketamine plus sub-effective dose lurasidone (0.1 mg/kg). However, when the scketamine was given sub-effective dose lurasidone plus sub-effective dose rapastinel, the animals explored the novel object significantly more compared to the familiar object (P<0.001; data not shown). The DI showed a significant interaction between groups (F_{4, 43}=10.04;***P<0.001; FIG. 3). Scketamine plus sal-, scketamine plus rapastinel (0.3 mg/kg)-, and scketamine plus lurasidone (0.1 mg/kg)-treated mice showed significant reductions in the DI compared to saline controls (***P<0.001). The effect of the combination of sub-effective dose rapastinel and lurasidone was not significantly different from that of the effective doses of either drug, given alone.

The combination of sub-effective dose rapastinel (0.3 mg/kg) plus sub-effective dose lurasidone (0.1 mg/kg) significantly reversed the decrease in the DI produced by scketamine (###P<0.001; FIG. 3). Rapastinel potentiated the atypical antipsychotic/antidepressant drug, lurasidone, to restore NOR in (sc) ketamine-treated mice.

Total Exploration Times:

No significant effect was observed between groups in the total exploration times (acquisition trial+retention trial). Mice from all treatment groups spent almost equal times exploring in the acquisition and retention trials (sal+sal=77.5±6.7; subchronic ketamine=86.3±5.2; subchronic ketamine+subeffective dose rapastinel=73.2±4.7; subchronic ketamine+subeffective dose lurasidone=71.5±6.2; subchronic ketamine+subeffective dose rapastinel+subeffective dose lurasidone=78.5±5.)

Examples 1-3 study strongly suggest that rapastinel, an NMDAR glycine site functional partial agonist, (i) did not induce NOR deficit; (ii) significantly prevented acute ketamine-induced NOR deficit and reversed the deficit in NOR produced by subchronic administration of the NMDAR antagonists, ketamine and PCP; and (iii) when administered at sub-effective dose, combined with antipsychotic/antidepressant sub-effective dose lurasidone restored NOR in scketamine-treated mice. Since drugs such as lurasidone which are ameliorative in the scNMDAR-model of cognitive impairment in schizophrenia are also effective to improve cognitive impairment in some patients with schizophrenia, rapastinel may also have some direct cognitive benefits in patients with schizophrenia or mood disorder.

### Example 4—Synthesis of (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide (Compound A)

The following reaction sequence was used (Scheme A) to synthesize (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide

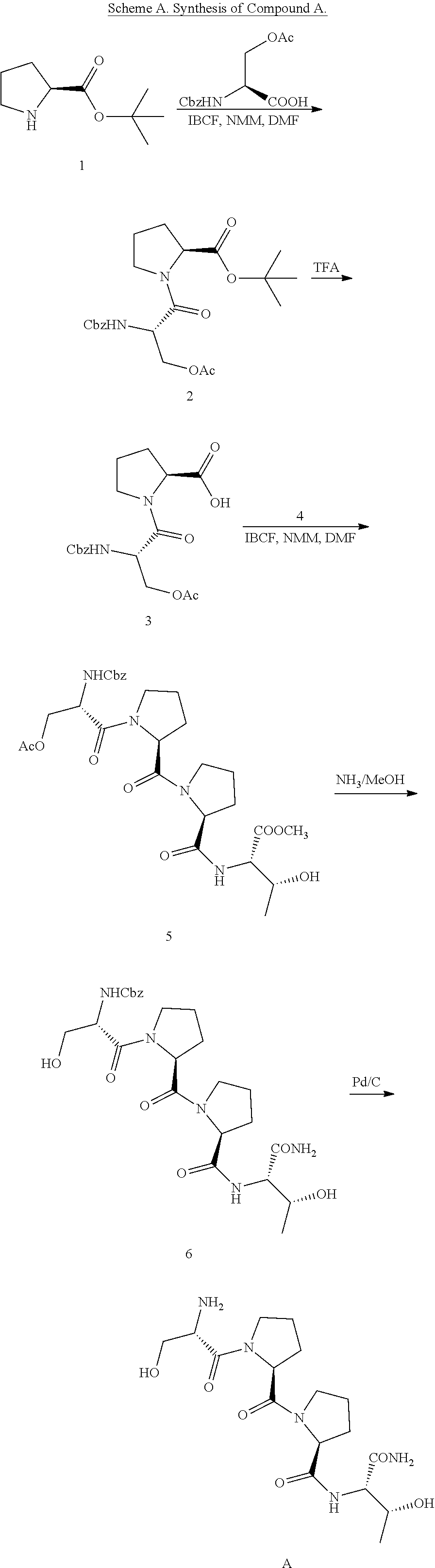

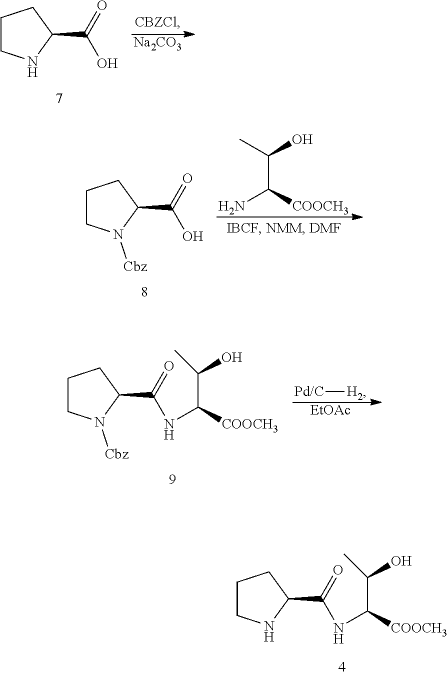

### Synthesis of (S)-tert-butyl 1-((S)-3-acetoxy-2-(benzyloxycarbonylamino)-propanoyl)-pyrrolidine-2-carboxylate (2)

(S)-3-Acetoxy-2-(benzyloxycarbonylamino)-propanoic acid (1.5 g, 5.33 mmol) was dissolved in CH2Cl2 (15 mL). N-Methylmorpholine (NMM) (0.64 mL, 5.87 mmol) and isobutyl chloroformate (IBCF) (0.72 mL, 6.12 mmol) were added at −15° C. and stirred for 30 minutes under inert atmosphere. A mixture of (S)-tert-butyl pyrrolidine-2-carboxylate (1) (998 mg, 5.87 mmol) and NMM (0.64 mL, 5.87 mmol) in DMF (5 mL) were added drop wise to the reaction mixture and stirring was continued for another 3 h at RT. The reaction mixture was diluted with DCM (200 mL), washed with water (50 mL), citric acid solution (10 mL) and brine (10 mL). The separated organic layer was dried over anhydrous Na2SO4 and concentrated under reduced pressure. The obtained crude residue was purified by silica gel column chromatography eluting with 30% EtOAc/Hexane to afford compound 2 (1.6 g, 69.5%).

¹H-NMR: (200 MHz, DMSO-d6): δ 7.81-7.76 (d, J=20.5 Hz, 1H), 7.35-7.30 (m, 5H), 5.03-4.97 (m, 2H), 4.61-4.55 (m, 1H), 4.32-4.16 (m, 2H), 4.08-3.87 (m, 2H), 3.65-3.59 (m, 1H), 2.21-2.11 (m, 2H), 1.98 (s, 3H), 1.91-1.75 (m, 2H), 1.37 (s, 9H); Mass m/z: 435.0 [M++1].

### Synthesis of (S)-1-((S)-3-acetoxy-2-(benzyloxycarbonylamino)-propanoyl)-pyrrolidine-2-carboxylic acid (3)

To a solution of compound 2 (1 g, 2.30 mmol) in CH₂Cl₂ (5 mL) was added 20% TFA-DCM (10 mL) and stirred at RT for 2 h. The reaction mixture was diluted with water (10 mE) and extracted with EtOAc (2×15 mL). The organic layer was dried over anhydrous Na₂SO₄ and concentrated under reduced pressure to yield compound 3 (800 mg, 92%).

¹H-NMR: (200 MHz, DMSO-d₆): δ 12.58 (br s, 1H), 7.81-7.77 (d, J=8.0 Hz, 1H), 7.35-7.27 (m, 5H), 5.04-4.96 (m, 2H), 4.66-4.60 (m, 1H), 4.32-4.24 (m, 2H), 4.04-3.86 (m, 1H), 3.66-3.59 (t, J=12.6 Hz, 2H), 2.17-2.07 (m, 3H), 1.98-1.80 (m, 4H); Mass m/z: 379.0 [M⁺+1].

### Synthesis of (2S,3R)-methyl 2-((S)-1-((S)-1-((R)-3-acetoxy-2-(benzyloxycarbonylamino)-propanoyl)-pyrrolidine-2-carbonyl)pyrrolidine-2-carboxamido)-3-hydroxybutanoate (5)

Compound 3 (1.0 g, 2.64 mmol) was dissolved in CH₂Cl₂ (10 mL), NMM (0.32 g, 3.17 mmol) and IBCF (0.41 g, 3.04 mmol) were added to the reaction mixture at −15° C. and stirred for 30 minutes under inert atmosphere. A mixture of (2S,3R)-methyl 3-hydroxy-2-((S)-pyrrolidine-2-carboxamido)-butanoate (4) (0.73 g, 3.17 mmol) and NMM (0.35 mL) in DMF (3 mL) were added drop wise to the reaction mixture at −15° C. and stirring was continued for another 3 h at RT. The reaction mixture was diluted with DCM (200 mL), washed with water (20 mL), citric acid solution (2×20 mL) and brine (2×50 mL). The separated organic layer was dried over anhydrous Na₂SO₄ and concentrated under reduced pressure. The crude residue obtained was purified by silica gel column chromatography eluting with 5% CH₃OH/EtOAc to afford compound (5) (0.29 g, 19%).

¹H-NMR: (500 MHz, DMSO-d₆): δ 7.83-7.81 (m, 1H), 7.72-7.70 (m, 1H), 7.36-7.35 (m, 5H), 5.07-5.01 (m, 2H), 4.99-4.93 (m, 1H), 4.58 (s, 1H), 4.50-4.48 (m, 1H), 4.26-4.22 (m, 2H), 4.07-4.00 (m, 2H), 3.89-3.86 (m, 1H), 3.61-3.55 (m, 5H), 3.53 (s, 1H), 3.39 (s, 1H), 2.12 (s, 1H), 1.98 (s, 3H), 1.94-1.83 (m, 4H), 1.81-1.80 (m, 3H), 1.05 (d, J=6.5 Hz, 3H). Mass m/z: 591.0 [M⁺+1].

### Synthesis of benzyl-(R)-1-((S)-2-((S)-2-((2S,3R)-1-(aminooxy)-3-hydroxy-1-oxobutan-2-ylcarbamoyl)-pyrrolidine-1-carbonyl)-pyrrolidin-1-yl)-3-hydroxy-1-oxopropan-2-ylcarbamate (6)

A solution of methanolic ammonia (3 mL) was added to compound 5 (0.28 g, 0.47 mmol) and stirred at RT for 18 h. The volatiles were evaporated under reduced pressure to afford compound 6 (0.21 g, 82.3%).

¹H-NMR: (500 MHz, DMSO-d₆): δ 7.38-7.31 (m, 5H), 7.26 (s, 1H), 7.10-7.03 (m, 2H), 6.65 (br s, 1H), 5.04-5.01 (m, 2H), 4.98-4.84 (m, 1H), 4.76-4.75 (m, 1H), 4.61 (s, 1H), 4.38-4.31 (m, 2H), 4.02-4.00 (m, 2H), 3.77-3.74 (m, 1H), 3.67-3.56 (m, 3H), 3.44-3.37 (m, 2H), 2.14-1.86 (m, 8H), 1.01-1.00 (m, 3H).

Mass m/z: 550 [M⁺+1].

Synthesis of (S)—N-((2S,3R)-1-amino-3-hydroxy-1-oxobutan-2-yl)-1-((S)-1-((S)-2-amino-3-hydroxypropanoyl)-pyrrolidine-2-carbonyl)-pyrrolidine-2-carboxamide (Compound A): To a solution of compound 6 (0.21 g, 0.39 mmol) in methanol (5 mL) was added 10% Pd/C (30 mg) and the reaction mixture was stirred under hydrogen atmosphere for 2 h. The reaction mixture was filtered over celite, solvent was evaporated in vacuo, and the crude residue obtained was triturated with diethyl ether to yield A (130 mg, 83.3%).

¹H-NMR: (500 MHz, DMSO-d₆) (Rotamers): δ 7.39 (d, J=8.0 Hz, 1H), 7.08-7.03 (m, 2H), 6.65 (br s, 1H), 4.89-4.85 (m, 1H), 1.61-1.59 (m, 1H), 4.39-4.38 (m, 1H), 4.02-4.00 (m, 2H), 3.68-3.52 (m, 4H), 3.43-3.36 (m, 2H), 3.22-3.10 (m, 2H), 2.19-2.13 (m, 1H), 2.07-1.98 (m, 1H), 1.93-1.81 (m, 5H), 1.75 (s, 2H), 1.01-1.00 (m, 3H).

LCMS m/z: 400.2 [M⁺+1].

HPLC Purity: 99.27%.

Synthesis of (S)-1-(benzyloxycarbonyl) pyrrolidine-2-carboxylic acid (8): To a stirred solution of (S)-pyrrolidine-2-carboxylic acid (7) (2.0 g, 17.39 mmol) in THF: H₂O (20 mL, 1:1) were added Na₂CO₃ (2.76 g, 26.08 mmol) and Cbz-Cl (3.54 g, 20.80 mmol) and stirred at RT for 18 h. The reaction mixture was washed with EtOAc (10 mL) and the aqueous layer was acidified with 3N HCl and extracted with EtOAc (2×20 mL). The combined organic layer was washed with brine, dried over anhydrous Na₂SO₄ and concentrated under reduced pressure to yield compound 8 (3.0 g, 69.7%).

¹H-NMR: (500 MHz, DMSO-d₆): δ 12.62 (br s, 1H), 7.36-7.22 (m, 5H), 5.12-5.00 (m, 2H), 4.24-4.15 (dd, J=5.0, 36.0 Hz, 1H), 3.46-3.31 (m, 2H), 2.25-2.15 (m, 1H), 1.94-1.79 (m, 3H).

Mass m/z: 250.0 [M⁺+1].

### Synthesis of (S)-benzyl 2-((2S,3R)-3-hydroxy-1-methoxy-1-oxobutan-2-ylcarbamoyl)pyrrolidine-1-carboxylate (9)

Compound 8 (5.0 g, 20.08 mmol) was dissolved in CH₂Cl₂ (50 mL), NMM (2.43 mE, 22.08 mmol) and IBCF (2.74 mL, 23.09 mmol) were added and stirred at −15° C. for 30 minutes under inert atmosphere. A mixture of (2S,3R)-methyl 2-amino-3-hydroxybutanoate (2.93 g, 22.08 mmol) and NMM (2.43 mL, 22.08 mmol) in DMF (15 mL) were added drop wise at −15° C. The resultant reaction mixture was stirred at RT for 3 h. It was diluted with DCM (200 mL) and the organic layer was washed with water (50 mL), brine (50 mL), dried over anhydrous Na₂SO₄ and concentrated under reduced pressure. The obtained crude was purified by silica gel column chromatography eluting with 30% EtOAc/Hexane to afford compound 9 (3.1 g, 42%).

¹H-NMR: (500 MHz, DMSO-d₆)(Rotamers): δ 7.98-7.94 (m, 1H), 7.35-7.27 (m, 5H), 5.09-4.94 (m, 3H), 4.44 (dd, J=5.5, 8.5 Hz, 1H), 4.29-4.27 (m, 1H), 4.12 (s, 1H), 3.62 (s, 3H), 3.44-3.30 (m, 2H), 2.20-2.08 (m, 1H), 1.87-1.78 (m, 3H), 1.08-0.94 (2 d, 3H).

Mass m/z: 365.0 [M⁺+1].

### Example 5—Synthesis of (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide (Compound B)

The following reaction sequence was used (Scheme B) to synthesize (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide:

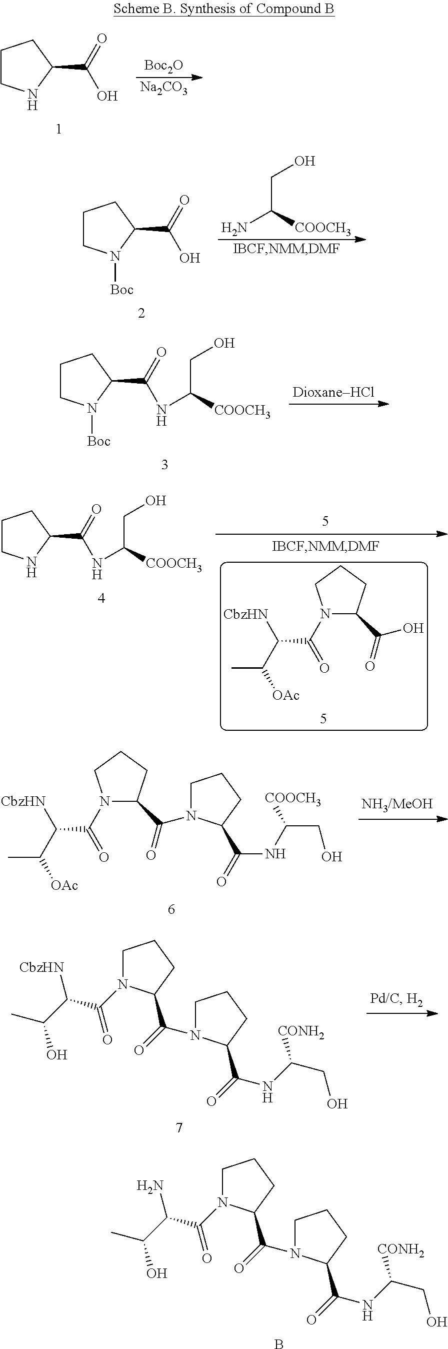

### Synthesis of (S)-1-(tert-butoxycarbonyl)-pyrrolidine-2-carboxylic acid (2)

To an ice cold stirred solution of (S)-pyrrolidine-2-carboxylic acid (1) (3.0 g, 26.08 mmol) in THF:H₂O (60 mL, 1:1) were added Na₂CO₃ (5.52 g, 52.16 mmol), Boc₂O (6.25 g, 26.69 mmol) and stirred at RT for 16 h. The reaction mixture was diluted with water and washed with EtOAc (50 mL). The aqueous layer was acidified with 2N HCl and extracted with EtOAc (2×100 mL). The combined organic layer was dried over anhydrous Na₂SO₄ and concentrated under reduced pressure to yield the (S)-1-(tert-butoxycarbonyl)-pyrrolidine-2-carboxylic acid (2) (4.8 g, 86%).

¹H-NMR: (500 MHz, DMSO-d₆): δ 12.49 (br s, 1H), 4.08-4.03 (m, 1H), 3.36-3.24 (m, 2H), 2.22-2.11 (m, 1H), 1.87-1.76 (m, 3H), 1.39 (s, 9H).

Mass m/z: 216.0 [M⁺+1].

### Synthesis of (S)-tert-butyl 2-((S)-3-hydroxy-1-methoxy-1-oxopropan-2-ylcarbamoyl)-pyrrolidine-1-carboxylate (3)

Compound 2 (2.0 g, 9.00 mmol) was dissolved in CH₂Cl₂ (10 mL) cooled to −15° C., NMM (1.12 mL, 10.2 mmol) and IBCF (1.26 mL, 1.15 mmol) were added and stirred at 0° C. for 20 minutes. A mixture of (S)-methyl 2-amino-3-hydroxypropanoate (1.59 g, 10.2 mmol) and NMM (1.12 mL) in DMF (3 mL) were added drop wise at −15° C. and the resultant reaction mixture was stirred at RT for 1 h. It was diluted with DCM (200 mL), water (50 mL) and washed with 2N HCl (20 mL) and brine (2×50 mL). The separated organic layer was dried over anhydrous Na₂SO₄ and concentrated under reduced pressure. The crude residue obtained was purified by silica gel column chromatography eluting with 20% EtOAc/Hexane to afford compound 3 (2.3 g) as a syrup.

Mass m/z: 317.0 [M⁺+1].

### Synthesis of (S)-methyl 3-hydroxy-2-((S)-pyrrolidine-2-carboxamido) propionate (4)

(S)-Tert-butyl-2-((S)-3-hydroxy-1-methoxy-1-oxopropan-2-ylcarbamoyl)-pyrrolidine-1-carboxylate (3) (500 mg, 1.58 mmol) was dissolved in 1,4-dioxane (3 mL) and a HCl solution in dioxane (3.16 mL, 3.16 mmol) was added stirred at RT for 4 h. The volatiles were evaporated under reduced pressure to afford compound 4 (280 mg) as solid.

¹H-NMR: (200 MHz, DMSO-d₆): δ 9.99 (br s, 1H), 9.12-9.08 (m, 1H), 8.53 (br s, 1H), 5.48 (br s, 2H), 4.43-4.22 (m, 2H), 3.82-3.67 (m, 4H), 3.56 (s, 3H), 2.36-2.27 (m, 1H), 1.93-1.86 (m, 3H).

Mass m/z: 217.0 [M⁺+1].

### Synthesis of (S)-methyl 2-((S)-1-((S)-1-((2R,3S)-3-acetoxy-2-(benzyloxycarbonylamino)-butanoyl)-pyrrolidine-2-carbonyl)-pyrrolidine-2-carboxamido)-3-hydroxypropanoate (6)

(2S)-1-((2R)-3-acetoxy-2-(benzyloxycarbonylamino)-butanoyl)-pyrrolidine-2-carboxylic acid (5) (1.3 g, 2.62 mmol) was dissolved in CH₂Cl₂ (15 mL), NMM (0.43 mL) and IBCF (0.51 mL) was added at −10° C. and stirred for 30 minutes under inert atmosphere. A mixture of (S)-methyl-3-hydroxy-2-((S)-pyrrolidine-2-carboxamido)-propionate (4) (992 mg, 3.93 mmol) and NMM (0.43 mL) in DMF (5 mL) were added drop wise to the reaction mixture and stirring was continued for another 3 h at RT. The reaction mixture was diluted with DCM (200 mL), washed with water (20 mL), citric acid solution (2×20 mL) and brine (2×50 mL). The separated organic layer was dried over anhydrous Na₂SO₄ and concentrated under reduced pressure. The obtained crude material was purified by silica gel column chromatography eluting with 5% CH₃OH/CH₂Cl₂ to afford compound 6 (270 mg, 17.5%).

¹H-NMR: (500 MHz, DMSO-d₆): δ 8.13 (d, J=8.0 Hz, 1H), 7.74 (d, J=7.5 Hz, 1H), 7.38-7.31 (m, 5H), 5.08-4.96 (m, 3H), 4.85-4.82 (m, 1H), 4.56 (d, J=8.0 Hz, 1H), 4.44-4.42 (m, 2H), 4.27 (d, J=7.0 Hz, 1H), 4.10 (d, J=10.5 Hz, 2H), 3.81-3.78 (m, 1H), 3.72-3.70 (m, 1H), 3.61-3.59 (m, 3H), 3.54-3.50 (m, 2H), 2.16-2.14 (m, 1H), 2.05-2.01 (m, 1H), 1.90 (s, 3H), 1.87-1.86 (m, 3H), 1.85-1.84 (m, 3H), 1.21-1.20 (d, J=6.0 Hz, 3H).

Mass m/z: 591.0 [M⁺+1].

### Synthesis of Benzyl-(2R,3S)-1-((S)-2-((S)-2-((S)-1-(aminooxy)-3-hydroxy-1-oxopropan-2-ylcarbamoyl) pyrrolidine-1-carbonyl)pyrrolidin-1-yl)-3-hydroxy-1-oxobutan-2-ylcarbamate (7)

To a solution of compound 6 (250 g, 0.42 mmol) in CH₃OH (2 mL) was added MeOH—NH₃ (10 mL) and was stirred at RT for 16 h. The volatiles were evaporated under reduced pressure to afford compound 7 (190 mg, 84%).

¹H-NMR: (500 MHz, DMSO-d₆): δ 7.60 (d, J=7.5 Hz, 1H), 7.35-7.30 (m, 5H), 7.18 (d, J=7.0 Hz, 1H), 7.11-7.06 (m, 2H), 5.05-4.97 (m, 2H), 4.82-4.81 (m, 1H), 4.60-4.59 (m, 2H), 4.33-4.31 (m, 1H), 4.15-4.08 (m, 2H), 3.81-3.79 (m, 1H), 3.72-3.64 (m, 2H), 3.59-3.53 (m, 4H), 2.14 (s, 1H), 2.03 (d, J=9.0 Hz, 1H), 1.95-1.85 (m, 5H), 1.75 (s, 1H), 1.10 (d, J=6.5 Hz, 3H).

Mass m/z: 550.0 [M⁺+1].

### Synthesis of (S)—N—((S)-1-amino-3-hydroxy-1-oxopropan-2-yl)-1-((S)-1-((2S,3R)-2-amino-3-hydroxybutanoyl) pyrrolidine-2-carbonyl)-pyrrolidine-2-carboxamide (B)

To a solution of compound 7 (190 mg, 0.35 mmol) in methanol (5 mL) was added 10% Pd/C (50 mg) and the reaction mixture was stirred under hydrogen atmosphere for 2 h. The reaction mixture was filtered through a celite pad, solvent was evaporated in vacuo and the crude was purified by column chromatography on basic alumina using 0-5% CH₃OH in CH₂Cl₂ as eluent to yield compound B (130 mg, 73%).

¹H-NMR: (500 MHz, DMSO-d₆): δ 7.65-7.60 (m, 1H), 7.12-7.03 (m, 2H), 4.81 (br s, 1H), 4.58-4.57 (m, 1H), 4.49 (m, 1H), 4.38-4.19 (m, 1H), 4.10-4.06 (m, 1H), 3.69-3.62 (m, 2H), 3.59-3.56 (m, 4H), 3.49-3.45 (m, 2H), 3.37-3.26 (m, 2H), 2.19-2.15 (m, 1H), 2.09-1.99 (m, 1H), 1.95-1.84 (m, 5H), 1.75 (s, 1H), 1.06 (d, J=13.0 Hz, 3H).

LCMS m/z: 400.8 [M⁺+1].

HPLC Purity: 97.71%.

### Example 6—Synthesis of (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide (Compound C)

The following reaction sequence was used (Scheme C) to synthesize (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide

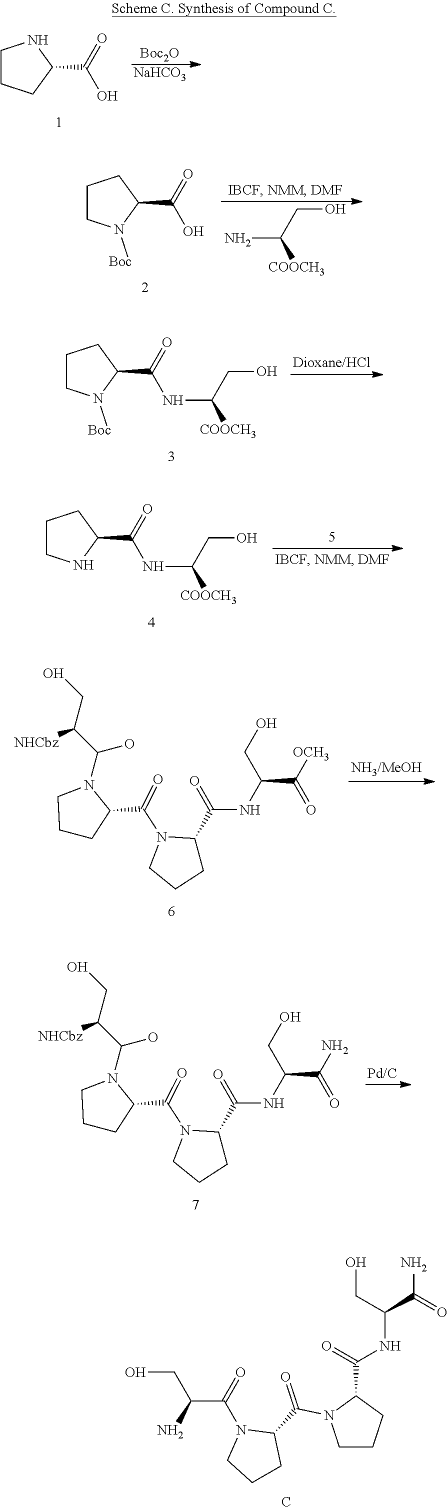

### Synthesis of (S)-1-(tert-butoxycarbonyl)-pyrrolidine-2-carboxylic acid (2)

To a stirred solution of (S)-pyrrolidine-2-carboxylic acid (3.0 g, 26.08 mmol) in THF:H₂O (60 mL, 1:1) at 0° C. were added Na₂CO₃ (5.52 g, 52.16 mmol) and Boc₂O (6.25 g, 26.69 mmol) and stirred at RT for 16 h. The reaction mixture was diluted with water and washed with EtOAc (50 mL). The aqueous layer was acidified with 2N HCl and extracted with EtOAc (2×50 mL). The combined organic layer was dried over anhydrous Na₂SO₄ and concentrated under reduced pressure to yield the (S)-1-(tert-butoxycarbonyl)-pyrrolidine-2-carboxylic acid 2 (4.8 g, 85.7%).

¹H-NMR: (500 MHz, DMSO-d₆): δ 12.49 (br s, 1H), 4.08-4.03 (m, 1H), 3.36-3.24 (m, 2H), 2.22-2.11 (m, 1H), 1.87-1.76 (m, 3H), 1.39 (s, 9H).

Mass m/z: 216.0 [M⁺+1].

### Synthesis of (S)-tert-butyl 2-((S)-3-hydroxy-1-methoxy-1-oxopropan-2-ylcarbamoyl) pyrrolidine-1-carboxylate (3)

Compound 2 (2.0 g, 9.00 mmol) was dissolved in CH₂Cl₂ (10 mL) cooled to −15° C., NMM (1.12 mL, 10.2 mmol) and IBCF (1.26 mL, 1.15 mmol) were added and stirred at 0° C. for 20 minutes. A mixture of (S)-methyl-2-amino-3-hydroxypropanoate (1.59 g, 10.2 mmol) and NMM (1.12 mL) in DMF (3 mL) were added drop wise at −15° C. The resultant reaction mixture was stirred at RT for 1 h. The reaction mixture was diluted with DCM (200 mL) and water (25 mL) and was washed with 2N HCl (20 mL) and brine (10 mL). The separated organic layer was dried over anhydrous Na₂SO₄ and concentrated under reduced pressure. The obtained crude material was purified by silica gel column chromatography eluting with 20% EtOAc/Hexane to afford compound 3 (2.3 g) as solid. Mass m/z: 317.0 [M⁺+1].

### Synthesis of (S)-methyl 3-hydroxy-2-((S)-pyrrolidine-2-carboxamido) propanoate (4)

To a solution of (S)-tert-butyl-2-((S)-3-hydroxy-1-methoxy-1-oxopropan-2-ylcarbamoyl) pyrrolidine-1-carboxylate 3 (500 mg, 1.58 mmol) in 1,4-dioxane (3 mL) was added a solution of HCl in dioxane (3.16 mL, 3.16 mmol) and stirred at RT for 4 h. The volatiles were evaporated under reduced pressure to afford compound 4 (280 mg) as solid.

¹H-NMR: (200 MHz, DMSO-d₆): δ 9.99 (br s, 1H), 9.12-9.08 (m, 1H), 8.53 (br s, 1H), 5.48 (br s, 2H), 4.43-4.22 (m, 2H), 3.82-3.67 (m, 4H), 3.56 (s, 3H), 2.36-2.27 (m, 1H), 1.93-1.86 (m, 3H).

Mass m/z: 217.0 [M⁺+1].

### Synthesis of (S)-methyl 2-((S)-1-((S)-1-((S)-2-(benzyloxycarbonylamino)-3-hydroxypropanoyl)-pyrrolidine-2-carbonyl)-pyrrolidine-2-carboxamido)-3-hydroxypropanoate (6)

(S)-1-((S)-3-Acetoxy-2-(benzyloxycarbonylamino)-propanoyl)-pyrrolidine-2-carboxylic acid (5) (400 mg, 1.05 mmol) was dissolved in CH₂Cl₂ (2 mL), NMM (0.13 mL) and IBCF (0.14 mL) were added at −15° C. and stirred for 30 minutes under inert atmosphere. A mixture of (S)-methyl-3-hydroxy-2-((S)-pyrrolidine-2-carboxamido)-propanoate hydrochloride (4) (293 mg, 1.16 mmol) and NMM (0.13 mL) in DMF (2 mL) were added drop wise to the reaction mixture and stirring was continued for another 3 h at RT. The reaction mixture was diluted with DCM (200 mL), washed with water (20 mL) and brine (10 mL). The separated organic layer was dried over anhydrous Na₂SO₄ and concentrated under reduced pressure. The obtained crude material was purified by silica gel column chromatography eluting with 5% CH₃OH/CH₂Cl₂ to afford compound 6 (80 mg, 13%).

¹H-NMR: (500 MHz, DMSO-d₆): δ 8.09 (d, J=7.5 Hz, 1H), 7.71 (d, J=8.0 Hz, 1H), 7.36-7.31 (m, 6H), 5.07-4.99 (m, 3H), 4.59-4.58 (m, 2H), 4.41-4.40 (m, 1H), 4.29-4.24 (m, 3H), 3.86 (t, J=9.5 Hz, 1H), 3.72-3.68 (m, 1H), 3.64-3.57 (m, 3H), 3.40-3.38 (m, 3H), 2.14-2.01 (m, 2H), 1.98 (s, 3H), 1.90-1.80 (m, 6H).

Mass m/z: 535.0 [M⁺+1].

### Synthesis of Benzyl-(S)-1-((S)-2-((S)-2-((S)-1-amino-3-hydroxy-1-oxopropan-2-ylcarbamoyl) pyrrolidine-1-carbonyl) pyrrolidin-1-yl)-3-hydroxy-1-oxopropan-2-ylcarbamate (7)

To a solution of (S)-methyl-2-((S)-1-((S)-1-((S)-2-(benzyloxycarbonylamino)-3-hydroxypropanoyl)-pyrrolidine-2-carbonyl)-pyrrolidine-2-carboxamido)-3-hydroxypropanoate (6) (60 mg, 1.04 mmol) in MeOH was added MeOH—NH₃ (3 mL) was stirred at RT for 16 h. The volatiles were evaporated under reduced pressure to afford compound 7 (30 mg, 55%).

¹H-NMR: (500 MHz, DMSO-d₆): δ 7.60 (d, J=7.5 Hz, 1H), 7.36-7.31 (m, 6H), 7.11-7.06 (m, 2H), 5.04-4.98 (m, 2H), 4.82-4.74 (m, 2H), 4.61-4.59 (m, 1H), 4.36-4.30 (m, 2H), 4.10-4.07 (m, 1H), 3.67-3.65 (m, 2H), 3.59-3.55 (m, 6H), 3.44-3.40 (m, 2H), 1.95-1.92 (m, 6H).

Mass m/z: 520.0 [M⁺+1].

### Synthesis of (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide (C)

Benzyl-(S)-1-((S)-2-((S)-2-((S)-1-amino-3-hydroxy-1-oxopropan-2-ylcarbamoyl) pyrrolidine-1-carbonyl) pyrrolidin-1-yl)-3-hydroxy-1-oxopropan-2-ylcarbamate 7 (300 mg, 0.57 mmol) was dissolved in methanol (8 mL), 10% Pd/C (50 mg) was added and reaction mixture was stirred under hydrogen atmosphere for 2 h. The reaction mixture was filtered and the filtrate was concentrated under reduced pressure to yield compound C (150 mg, 68%).

¹H-NMR: (500 MHz, DMSO-d₆) (Rotamers): δ 7.62 (d, J=8.0 Hz, 1H), 7.24 (br s, 1H), 7.14-7.07 (m, 2H), 4.87-4.82 (m, 2H), 4.59-4.57 (m, 1H), 4.37-4.31 (m, 2H), 4.11-4.07 (m, 2H), 3.70-3.39 (m, 8H), 2.17-2.01 (m, 2H), 1.95-1.79 (m, 6H).

LCMS m/z: 386.4 [M⁺+1].

HPLC Purity: 98.45%.

### Example 7—Synthesis of N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide (Compound D & E)

The following reaction sequence was used (Scheme D) to synthesize N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzsaylpyrrolidine-2-carboxamide (Compound D & E):

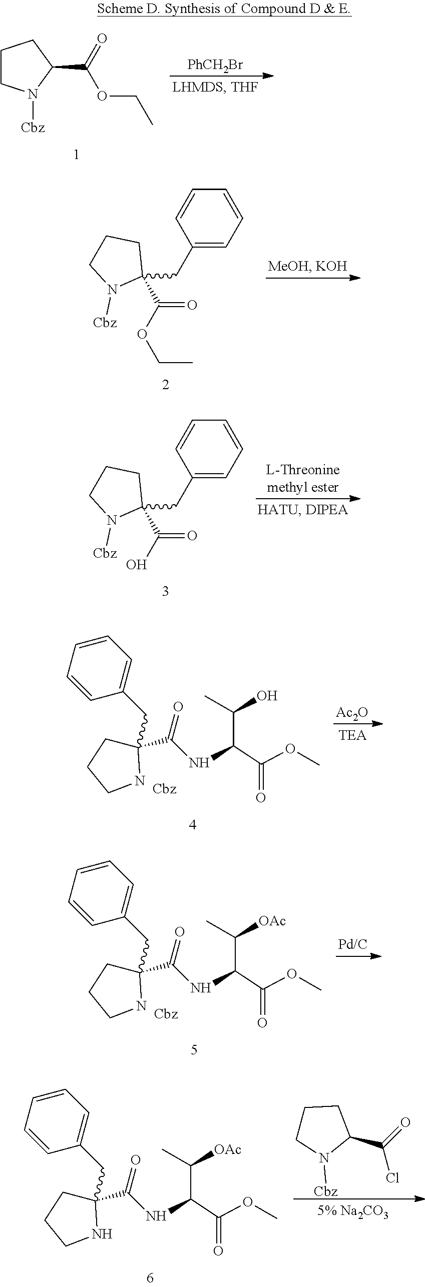

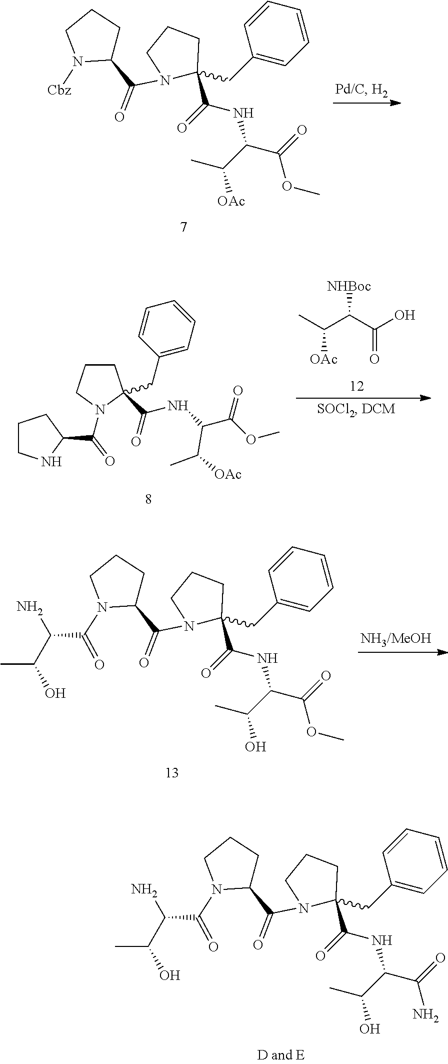

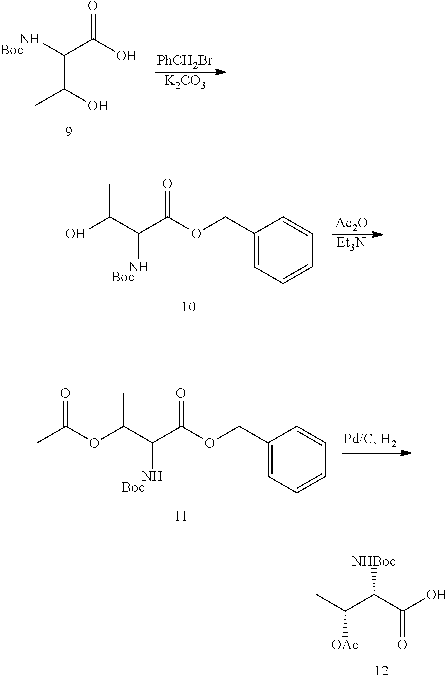

### Synthesis of 1-Benzyl 2-ethyl 2-benzylpyrrolidine-1,2-dicarboxylate (2)

To a solution of (S)-1-benzyl-2-ethyl-pyrrolidine-1, 2-dicarboxylate (1) (10 g, 36.10 mmol) in THF (150 mL) under inert atmosphere was added LiHMDS (1M in THF) (43.3 mL, 43.3 mmol) at −25° C. and stirred for 2 h. Benzyl bromide (5.17 mL, 43.26 mmol) was added drop wise at −25° C. to the reaction mixture. It was allowed to warm to RT and stirred for 2 h. The reaction mixture was cooled to 5° C., quenched with saturated NH₄Cl solution and the aqueous layer was extracted with EtOAc (2×200 mL). The combined organic extracts were dried over anhydrous Na₂SO₄ and concentrated under reduced pressure. The crude residue obtained was purified by silica gel column chromatography eluting with 5% EtOAc/hexane to afford compound 2 (13 g, 75%) as liquid.

¹H-NMR: (200 MHz, DMSO-d₆): δ 7.47-7.32 (m, 5H), 7.27-7.16 (m, 3H), 7.07-7.04 (m, 2H), 5.29-5.06 (m, 2H), 4.16-3.89 (m, 2H), 3.57-3.33 (m, 2H), 3.02-2.78 (m, 2H), 2.13-1.89 (m, 2H), 1.56-1.51 (m, 1H), 1.21-1.04 (m, 3H), 0.93-0.79 (m, 1H).

Mass m/z: 368.2 [M⁺+1].

### Synthesis of 2-benzyl-1-(benzyloxycarbonyl)-pyrrolidine-2-carboxylic acid (3)

To a stirred solution of compound 2 (8.0 g, 21.79 mmol) in CH₃OH (20 mL) was added 2N aqueous KOH (20 mL) and heated up to 100° C. and stirred for 16 h. The volatiles were evaporated under reduced pressure. The residue obtained was diluted with ice cold water (50 mL) and washed with ether (50 mL). The aqueous layer was acidified to pH-2 using HCl solution and extracted with EtOAc (2×100 mL). The combined organic layer was dried over anhydrous Na₂SO₄ and concentrated under reduced pressure to afford compound 3 (6 g, 81%) as an off white solid.

¹H-NMR: (200 MHz, DMSO-d₆): δ 12.71 (br s, 1H), 7.40-7.30 (m, 5H), 7.25-7.19 (m, 3H), 7.07-7.00 (m, 2H), 5.27-5.02 (m, 2H), 3.59-3.32 (m, 2H), 3.02-2.83 (m, 2H), 2.13-1.91 (m, 2H), 1.58-1.49 (m, 1H), 0.90-0.77 (m, 1H).

Mass m/z: 340.1 [M⁺+1].

### Synthesis of Benzyl-2-benzyl-2-((2S,3R)-3-hydroxy-1-methoxy-1-oxobutan-2-ylcarbamoyl)-pyrrolidine-1-carboxylate (4)

To a suspension of compound 3 (1.0 g, 2.94 mmol), L-threonine methyl ester (471 mg, 3.53 mmol) in DMF (20 mL) was added HATU (1.12 g, 2.94 mmol) and DIPEA (1.58 mL, 8.84 mmol) at 5° C. The reaction mixture was stirred at RT for 16 h. It was diluted with EtOAc (150 mL) and washed with water (2×30 mL). The organic layer was washed with brine, dried over Na₂SO₄, concentrated and purified by silica gel column chromatography 50% EtOAc/Hexane as eluent to yield compound 4 (1.0 g, 74%).

¹H-NMR: (200 MHz, DMSO-d₆): δ 7.62-7.59 (m, 1H), 7.44-7.31 (m, 5H), 7.21-7.18 (m, 3H), 7.06-6.99 (m, 2H), 5.25-5.24 (m, 1H), 5.12-4.94 (m, 2H), 4.30 (s, 1H), 4.15-4.08 (m, 1H), 3.66-3.64 (m, 3H), 3.63-3.49 (m, 2H), 3.14 (s, 1H), 2.89 (s, 1H), 2.09-2.02 (m, 2H), 1.56-1.51 (m, 1H), 1.09-0.98 (m, 4H).

Mass m/z: 455.1 [M⁺+1], 477.3 [M+Na].

### Synthesis of Benzyl-2-((2S,3R)-3-acetoxy-1-methoxy-1-oxobutan-2-ylcarbamoyl)-2-benzylpyrrolidine-1-carboxylate (5)

Compound 4 (3 g, 6.60 mmol) was dissolved in THF (30 mL), Et₃N (1.11 mL, 7.92 mmol) and Ac₂O (742 mg, 7.26 mmol) were added at RT. The reaction mixture was stirred at RT for 2 h. The volatiles were evaporated under reduced pressure and the residue obtained was diluted with CH₂Cl₂ and washed with dilute HCl. The combined organic extracts were dried over Na₂SO₄ and concentrated under reduced pressure. The crude residue was purified by column chromatography using 30% EtOAc/Hexane as eluent to yield compound 5 (2.5 g, 76%).

¹H-NMR: (500 MHz, DMSO-d₆) (Rotamers): δ 8.15-7.71 (m, 1H), 7.42-7.04 (m, 10H), 5.30-5.19 (m, 2H), 5.11-5.09 (m, 1H), 4.99-4.93 (m, 1H), 4.67-4.62 (m, 1H), 3.66-3.64 (m, 3H), 3.55-3.46 (m, 2H), 3.38-3.35 (m, 1H), 2.88-2.69 (m, 1H), 2.17-2.00 (m, 2H), 1.98-1.92 (m, 3H), 1.56-1.46 (m, 1H), 1.23-1.17 (m, 3H), 1.02-0.86 (m, 1H).

LCMS m/z: 497.4 [M⁺+1].

### Synthesis of (2S,3R)-methyl 3-acetoxy-2-(2-benzylpyrrolidine-2-carboxamido)-butanoate (6)

To a stirring solution of compound 5 (4 g, 8.06 mmol) in ethanol (50 mL) was added 10% Pd/C (1.2 g) and the reaction mixture was stirred under H₂ atmosphere (balloon pressure) for 4 h. It was filtered through celite pad and the filtrate was concentrated under reduced pressure to yield compound 6 (2.2 g, 75%).

¹H-NMR: (500 MHz, DMSO-d₆) (Rotamers): δ 8.22-8.17 (m, 1H), 7.24-7.16 (m, 5H), 5.17 (t, J=11.5 Hz, 1H), 4.48-4.42 (m, 1H), 3.60-3.54 (s, 3H), 3.20 (t, J=13.5 Hz, 1H), 3.06-2.97 (m, 1H), 2.82-2.68 (m, 3H), 2.08-2.02 (m, 1H), 1.89 (s, 3H), 1.72-1.51 (m, 3H), 1.10 (2 d, 3H).

LCMS m/z: 363 [M⁺+1], 385 [M+Na].

### Synthesis of (S)-benzyl 2-(2-((2S,3R)-3-acetoxy-1-methoxy-1-oxobutan-2-ylcarbamoyl)-2-benzylpyrrolidine-1-carbonyl) pyrrolidine-1-carboxylate (7)

To a stirred solution of compound 6 (1 g, 2.76 mmol) and Na₂CO₃ (732 mg, 6.90 mmol) in CH₂Cl₂:H₂O (20 mL, 1:1) was added a solution of acid chloride [To a solution of (S)-1-(benzyloxycarbonyl) pyrrolidine-2-carboxylic acid (825 mg, 3.31 mmol) in CH₂Cl₂ (20 mL) was added SOCl₂ (0.60 mL) drop wise at 0° C. and was refluxed for 2 h. The volatiles were removed under reduced pressure to yield (S)-benzyl 2-(chlorocarbonyl) pyrrolidine-1-carboxylate] in CH₂Cl₂ and the reaction mixture was stirred at RT for 2 h. The volatiles were evaporated under reduced pressure. The residue was diluted with CH₂Cl₂ (100 mE), filtered and the filtrate was concentrated under vacuum. The crude residue was purified by column chromatography using 60% EtOAc/Hexane as eluent to afford compound 7 (750 mg, 45%).

¹H-NMR: (500 MHz, DMSO-d₆) (Rotamers): δ 7.36-7.23 (m, 8H), 7.15-7.12 (m, 3H), 5.21-5.15 (m, 2H), 5.04-4.92 (m, 1H), 4.57-4.50 (m, 2H), 3.88 (d, J=14.5 Hz, 1H), 3.65 (s, 3H), 3.54-3.46 (m, 3H), 3.21-3.13 (m, 1H), 3.02-2.90 (m, 2H), 2.19-2.02 (m, 4H), 1.97 (s, 3H), 1.89 (s, 1H), 1.77-1.65 (m, 1H), 1.17 (s, 2H), 1.06 (s, 2H).

Mass m/z: 594.1 [M⁺+1].

### Synthesis of (2S,3R)-methyl 3-acetoxy-2-(2-benzyl-1-((S)-pyrrolidine-2-carbonyl)-pyrrolidine-2-carboxamido) butanoate (8)

To a solution of compound 7 (200 mg, 0.336 mmol) in EtOAc (15 mL) was added 10% Pd/C (40 mg) was added under inert atmosphere and stirred for 12 h under H₂ atmosphere (balloon pressure). The reaction mixture was filtered through celite pad and concentrated under reduced pressure. The obtained residue was triturated with ether (10 mL) to afford compound 8 (125 mg, 81%) as solid.

¹H-NMR: (500 MHz, CDCl₃) (Rotamers): δ 7.88-7.87 (d, 1H, J=8.5), 7.30-7.26 (m, 2H), 7.24-7.21 (m, 1H), 7.13-7.12 (d, 2H, J=7), 5.44-5.43 (m, 1H), 4.76-4.74 (m, 1H), 3.94-3.92 (m, 1H), 3.84-3.81 (m, 1H), 3.75 (s, 3H), 3.50 (m, 1H), 3.26-3.12 (m, 3H), 2.90-2.88 (m, 1H), 2.23-2.15 (m, 4H), 2.04 (s, 3H), 1.87-1.77 (m, 5H), 1.27-1.24 (n, 3H).

Mass m/z: 460 (M+1).

### Synthesis of Benzyl-2-(tert-butoxycarbonylamino)-3-hydroxybutanoate (10)

To a solution of 2-(tert-butoxycarbonylamino)-3-hydroxybutanoic acid (3.0 g, 13.69 mmol) in DMF (50 mL) was added K₂CO₃ (3.73 g, 27.39 mmol) and stirred at RT for 15 min. (Bromomethyl)benzene (2.81 g, 16.43 mmol) was added and stirred at RT for 6 h. The reaction mixture was diluted with water (50 mL) and extracted with EtOAc (2×50 mL). The combined organic layer was washed with brine (50 mL), dried over anhydrous Na₂SO₄ and concentrated under reduced pressure. The crude material was purified by silica gel column chromatography using 20% EtOAc/hexane as eluent to afford benzyl 2-(tert-butoxycarbonylamino)-3-hydroxybutanoate 10 (2.8 g, 66%).

¹H-NMR: (500 MHz, DMSO-d₆): δ 7.37-7.30 (m, 5H), 6.60 (d, J=8.5 Hz, 1H), 5.18-5.08 (m, 2H), 4.76 (d, J=7 Hz, 1H), 4.08-4.00 (m, 2H), 1.38 (s, 9H), 1.09 (d, J=6.0 Hz, 3H).

Mass m/z: 310.0 [M⁺+1], 210 [M⁺-De Boc].

### Synthesis of benzyl-3-acetoxy-2-(tert-butoxycarbonylamino)-butanoate (11)

To a stirred solution of benzyl-2-(tert-butoxycarbonylamino)-3-hydroxybutanoate (2.8 g, 9.06 mmol) in THF (80 mL) was added Ac₂O (1.1 g, 10.87 mmol), Et₃N (1.51 mL, 10.87 mmol) and DMAP (280 mg) and stirred at RT for 15 min. The volatiles were removed under reduced pressure. The residue obtained was diluted with EtOAc (150 mL) and washed with cold 0.5N HCl solution (2×20 mL). The organic layer was washed with brine, dried over anhydrous Na₂SO₄ and concentrated under reduced pressure to afford 3-acetoxy-2-(tert-butoxycarbonylamino)-butanoate 11 (2.8 g, 88%).

¹H-NMR: (500 MHz, DMSO-d₆): δ 7.35-7.34 (m, 5H), 7.27-7.25 (d, J=8.5 Hz, 1H), 5.18-5.06 (m, 3H), 4.34-4.32 (m, 1H), 1.90 (s, 3H), 1.39 (s, 9H), 1.16 (d, J=3 Hz, 3H).

Mass m/z: 252 [M⁺+1-De Boc].

### Synthesis of (2S,3R)-3-acetoxy-2-(tert-butoxycarbonylamino)-butanoic acid (12)

Benzyl-3-acetoxy-2-(tert-butoxycarbonylamino) butanoate 11 (1.4 g, 3.98 mmol) was dissolved in EtOAc (40 mL), 10% Pd/C (600 mg) was added and reaction mixture was stirred under hydrogen atmosphere for 16 h. The reaction mixture was filtered over celite, solvent was evaporated in vacuo and the crude residue was triturated with hexane to yield (2S,3R)-3-acetoxy-2-(tert-butoxycarbonylamino) butanoic acid 12 (0.7 g, 70%).

¹H-NMR: (500 MHz, DMSO-d₆): δ 12.78 (br s, 1H), 6.94 (d, J=9.5 Hz, 1H), 5.16-5.14 (m, 1H), 4.17-4.15 (m, 1H), 1.95 (s, 3H), 1.39 (s, 9H), 1.10 (d, J=6.0 Hz, 3H). Mass m/z: 260.0 [M−1].

### Synthesis of (2S,3R)-methyl-3-acetoxy-2-(1-((S)-1-((2S,3R)-3-acetoxy-2-(tert-butoxycarbonyl-amino)-butanoyl)-pyrrolidine-2-carbonyl)-2-benzylpyrrolidine-2-carboxamido)-butanoate (13)

To a solution of compound (2S,3R)-3-acetoxy-2-(tert-butoxycarbonylamino)-butanoic acid 12 (199 mg, 0.76 mmol) in CH₂Cl₂ (6 mL) was under inert atmosphere were added IBCF (125 mg, 0.91 mmol) and NMM (154 mg, 1.52 mmol) at −15° C. and stirred for 1 h. A solution of (2S,3R)-methyl 3-acetoxy-2-(2-benzyl-1-((S)-pyrrolidine-2-carbonyl) pyrrolidine-2-carboxamido)-butanoate 8 (350 mg, 0.76 mmol) in DMF (2 mL) was added to the reaction mixture and stirred for 1 h at −15° C. The resultant reaction mixture was allowed to warm to RT and stirred for 19 h. The reaction mixture was extracted with EtOAc and the separated organic layer was washed with water (20 mL), followed by brine (20 mL), dried over Na₂SO₄ and concentrated under reduced pressure. The crude material was purified by preparative HPLC to afford compound 13 (100 mg, 20%).

¹H-NMR: (500 MHz, CD₃OD) (Rotamers): δ 7.30-7.24 (m, 3H), 7.15-7.13 (m, 2H), 4.62-4.55 (m, 2H), 4.29-3.97 (m, 1H), 3.98-3.79 (m, 4H), 3.75 (s, 3H), 3.62-3.22 (m, 2H), 3.23 (d, J=13.5 Hz, 1H), 3.00-2.95 (q, 1H), 2.37-2.31 (m, 1H), 2.23-2.10 (m, 2H), 2.02-1.88 (m, 3H), 1.46-1.28 (m, 2H), 0.97 (d, J=7.0 Hz, 6H).

### Synthesis of N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide (D & E)

A solution of compound 13 (100 mg, 0.153 mmol) in methanolic-NH₃ (10 mL) was stirred in a sealed tube at RT for 72 h. The reaction mixture was concentrated under reduced pressure. The obtained crude residue was washed with ether (2×2 mL) to afford a diastereomeric mixture of Compound D & E (85 mg). 85 mg of this mixture was further purified by chiral preparative HPLC to yield 15 mg each of Compound D and E.

¹H-NMR: (500 MHz, CD₃OD) (Rotamers): δ 7.33-7.26 (m, 3H), 7.16 (s, 2H), 4.55-4.54 (m, 1H), 4.39 (s, 1H), 4.14 (s, 1H), 4.01-3.98 (m, 1H), 3.91-3.71 (m, 3H), 3.59 (s, 2H), 3.25-3.16 (m, 1H), 3.04-3.00 (m, 1H), 2.33-2.10 (m, 3H), 2.01-1.91 (m, 2H), 1.86-1.80 (m, 1H), 1.46-1.44 (m, 1H), 1.34-1.29 (m, 1H), 1.25-1.19 (m, 3H), 0.99-0.97 (d, J=14.0 Hz, 3H).

Mass m/z: 503 [M+]; HPLC Purity: 98.1%.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A method of treating schizophrenia in a patient in need thereof, comprising administering to the patient:
an atypical antipsychotic; and
an NMDA modulator selected from the group consisting of rapastinel, (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

2. A method of treating bipolar disorder in a patient in need thereof, comprising administering to the patient:
an atypical antipsychotic; and
an NMDA modulator selected from the group consisting of rapastinel, (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

3. A method of treating a cognitive impairment disorder in a patient in need thereof, comprising administering to the patient an atypical antipsychotic; and an NMDA modulator selected from the group consisting of rapastinel, (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

4. The method of claim 3, wherein the cognitive impairment disorder is due to one or more of: deficit in cognitive ability, congenital defect, environmental factor(s), or drug induced.

5. The method of claim 4, wherein the cognitive impairment disorder is a learning disorder, autism, and/or dyslexia.

6. A method of treating major depressive disorder in a patient in need thereof, comprising administering to the patient
an atypical antipsychotic; and
an NMDA modulator from the group consisting of rapastinel, (S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl) pyrrolidine-2-carbonyl) pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

7. The method of claim 6, wherein the major depressive disorder is refractory.

8. The method of claim 1, wherein the atypical antipsychotic is selected from the group consisting of lurasidone, quetiapine, olanzapine, asenapine, risperidone, ziprasidone, clozapine, melperone, cariprazien, aripiprazole, pimavenserin, ITI-007, RP506, and reomxipride.

9. The method of claim 1, wherein the atypical antipsychotic is lurasidone.

10. The method of claim 1, wherein the NMDA modulator and the atypical antipsychotic are each administered at a dose that is sub-effective if administered alone.

11. The method of claim 1, wherein administering an NMDA modulator occurs substantially simultaneously with administration of the atypical antipsychotic.

12. The method of claim 1, wherein the NMDA modulator and the atypical antipsychotic are administered sequentially.

13. The method of claim 12, wherein the NMDA modulator is administered before the atypical antipsychotic.

14. The method of claim 12, wherein the NMDA modulator is administered after the atypical antipsychotic.

15. A method of substantially reversing or preventing cognitive impairment in a patient subchronically administered a NMDAR antagonist, comprising administering an sub-effective amount of an NMDA modulator and a sub-effective amount of an atypical antipsychotic to said patient.

16. The method of claim 15, wherein the NMDAR antagonist is ketamine.

17. The method of claim 16, wherein the NMDA modulator is rapastinel.

18. The method of claim 1, where in the NMDA modulator is N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide.

19-23. (canceled)
